(19) United States
(12) Patent Application Publication
(10) Pub. No.: US 2009/0030364 A1
(43) Pub. Date: Jan. 29, 2009

(54) Electroporation gene therapy gun system

(75) Inventors: John W. Harmon, Baltimore, MD (US); Guy Pierre Marti, Cockeysville, MD (US); Mark John Ferguson, Inverurie (GB)

Correspondence Address: BANNER & WITCOFF, LTD., 1100 13th STREET, N.W., SUITE 1200, WASHINGTON, DC, 20005-4051, US

(73) Assignee: The Johns Hopkins University, Baltimore, MD (US)

(21) Appl. No.: 11/883,554

(22) PCT Filed: Feb. 6, 2006
(86) PCT No.: PCT/US06/03916

§ 371 Date: May 13, 2008

Related U.S. Application Data
(60) Provisional application No. 60/650,154, filed on Feb. 4, 2005.

(51) Int. Cl. A61N 1/30

(52) U.S. Cl. 604/21; 604/20

(57) ABSTRACT

A cartridge for administration of a bioactive agent may include a plurality of chambers, including at least a first chamber and a second chamber. Each of the chambers may contain a component of a composition to be administered to a tissue site of a subject. For example, the first chamber may contain the bioactive agent and the second chamber may contain a liquid for suspending the bioactive agent prior to administration to the tissue site. An apparatus for administering or delivering a bioactive agent may include the cartridge and may further include an energy source for providing an electrical pulse to the tissue site. The apparatus may further cause mixing of the bioactive agent with the liquid in the cartridge. For example, the apparatus may impact the cartridge to result in mixing of the bioactive agent with the liquid. A method for combining a bioactive agent with a liquid forms a composition for delivering to a tissue site.

This application claims the benefit of U.S. Provisional application No. 60/650,154, filed Feb. 4, 2005, which is incorporated by reference herein in its entirety.

### BACKGROUND OF THE INVENTION

Effective administration of bioactive agents to a tissue site is important to the treatment or prophylaxis of various medical conditions in or around the tissue site as well as systemically. Often, administration of bioactive agents, such as drugs, medications, pharmaceuticals, topical agents, vaccines, etc., is hampered by various factors such as the condition of the tissue receiving the bioactive agent. For example, scarring at the tissue site or any pre-existing injury to the tissue site may impair absorption of the bioactive agent. Some tissues in need of bioactive agents may be inherently less than optimally absorbent.

Such bioactive agents may be desired at a tissue site for any number of conditions. For example, administration of chemotherapeutic agents for the treatment of tumors may be needed. There are many possible conditions in which administration of bioactive agents may be desired or necessary, such as in the treatment of infections with the administration of antibiotics, anti-viral agents or antifungal agents; the administration of agents in cosmetic therapy, or the administration of agents for enhancing wound healing, to name a few.

In one such condition, a tissue site may contain a wound and healing of the wound may be desired. Many wounds are non-healing, particularly in patients with pre-existing conditions that cause slowed wound healing such as diabetic patients, patients with decubitus ulcers (i.e., pressure sores), patients with peripherally located lesions (e.g., leg sores), burn patients who may require skin grafting, patients with slow healing or non-healing gastrointestinal ulcers, erosions, or fissures, etc. In addition, many therapeutic options for wound healing involve topical ointments or creams which may not be adequately absorbed.

Growth factors and cytokines are typically depleted in wounds with impaired healing. When growth factors are applied topically to cutaneous wounds, they are rapidly deactivated by proteases within and surrounding the wound tissue. Nucleic acids encoding growth factors can be delivered into wounds where the growth factors are expressed continuously within the wound tissue. Such treatment improves wound healing. Moreover, electroporation improves transfection efficiency. See PCT/US03/04143, filed on Dec. 29, 2003, published as WO 04/06047, the disclosure of which is expressly incorporated herein.

### SUMMARY OF THE INVENTION

The present invention provides a cartridge for delivery of a composition to a tissue of a subject. The cartridge may contain, for example, a first chamber comprising a bioactive agent, a second chamber comprising a liquid, an orifice through which the composition is administered, and a conductive portion for transmitting an electrical pulse. For example, the bioactive agent may include a macromolecule for treating a condition at the tissue of the subject.

In another embodiment, the cartridge may include at least one electrode operatively connected to the conductive portion of the cartridge. The conductive portion may also include electrical wires and may be operatively connected to electrode needles.

In another embodiment, the orifice of the cartridge may be in a needle through which the composition may be administered. In one example, the cartridge also includes electrode needles in which the needle may extend from the cartridge further than the electrode needles.

In another embodiment, an apparatus is provided for delivering a composition to a tissue of a subject including a cartridge, an energy source, a means for combining a bioactive agent and a liquid to form a composition, and at least one electrode for delivering an electric field to a tissue.

In another embodiment, the means for combining a bioactive agent and a liquid of the apparatus causes an increase in pressure in a chamber of the cartridge to modify a membrane separating chambers of the cartridge. The modification of the membrane may cause combining of the bioactive agent and the liquid.

In another embodiment, the energy source of the apparatus generates an electric field based on pre-set parameters. The apparatus may further comprise a battery or an electrical cord and may further include a foot control device for actuating an energy source.

In another embodiment, a method is provided for delivering a liquid composition to a tissue site in a subject. A separating element between a bioactive agent and a liquid in separate chambers is modified so that the bioactive agent and liquid mix to form a liquid composition. The liquid composition is administered to the tissue site. An electrical field is established at the tissue site.

### BRIEF DESCRIPTION OF THE DRAWINGS

FIG. 1 is a side/frontal perspective view of an example of a device according to one aspect of the present invention.

FIG. 2 is a side and rear perspective view of an example of a device according to one aspect of the present invention.

FIG. 3 is a perspective view of an example of a device according to one aspect of the present invention.

FIG. 4 is a schematic side elevation view of an example of a device according to one aspect of the present invention.

FIG. 5 is a cross sectional side view of an example of a device according to one aspect of the present invention.

FIG. 6 is a side and top perspective view of an example of a portable battery according to one aspect of the present invention.

FIG. 7 is a side and top perspective view of an example of a battery and cord according to one aspect of the present invention.

FIG. 8 is a side and frontal perspective view of an example of a cartridge device according to one aspect of the present invention.

FIG. 9 is a side and rear perspective view of an example of a cartridge device according to one aspect of the present invention.

FIG. 10 is a schematic side elevation view of an example of a cartridge device according to one aspect of the present invention.

FIG. 11 is a cross sectional side view of an example of a cartridge device according to one aspect of the present invention.

FIG. 12 is a side and frontal perspective view of an example of a partially assembled device according to one aspect of the present invention.

FIG. 13 is a schematic side elevation view of an example of an assembled device according to one aspect of the present invention.

FIG. 14 illustrates treatment of wounds in an animal model according to one aspect of the present invention.

FIGS. 15A-15I illustrate luciferase expression in cutaneous tissue treated with intradermal delivery of a bioactive agent according to one aspect of the present invention.

### DETAILED DESCRIPTION

A cartridge for delivery of a composition to a tissue of a subject includes at least a first chamber and a second chamber containing components of the composition to be administered or delivered to the subject. The first chamber contains a bioactive agent including, for example, a macromolecule that is administered to a tissue site for treating a variety of conditions including tumors, skin disorders, infections, wound healing, etc.

The cartridge may be used to administer a bioactive agent that comprises a peptide, protein, growth factor or cytokine for treating tumors (e.g., chemotherapeutic agents, dissolving agents, herbal remedies), treating a skin disorder including degenerative disorders of the skin, alopecia/hair loss, hypertrophic scars, inflammatory scars, keloids, etc. (e.g., antibiotics, antifungal agents, compounds treating degenerative skin disorders, compounds treating alopecia, growth factors, hydrocortisone or other steroids), treating infections (e.g., antibiotics, antifungal agents, anti-viral agents), inducing or enhancing an immune response (e.g., immunogenic antigen, vaccine, antibodies), treating a hormonal imbalance or disorder (e.g., thyroid hormone, thyroid stimulating hormone, insulin, Adrenocorticotropic Hormone (ACTH)), cytokines, medications, drugs, factors, etc. In addition, the macromolecule may be a tagged macromolecule or a reporter that may be used in experimental research. For example, the macromolecule may be tagged with any number of fluorescent molecules and detected based on the fluorescent tag. Any of these bioactive agents or macromolecules may be in a dry or wet form. Thus, any desired substance or combination of substances may be applied to a tissue site. The tissue site may include any tissue site in need of treatment such as but not limited to skin, muscle, stomach, esophagus, small bowel, colon, spleen, liver, salivary gland, bone, tendon, nerves, bladder, etc. Other medicaments useful at a particular target tissue may be administered in this way.

The bioactive agent may be maintained at room temperature while in the cartridge. Optionally, the bioactive agent may be refrigerated, frozen or heated while in the cartridge.

A user may select a desired cartridge based on the bioactive agent contained in the cartridge. Based on the nature of the condition to be treated at the tissue site, the user may select a corresponding cartridge containing the desired drug, medication, factor, cytokine, nucleic acid, etc. For example, if wound healing is desired, a user may select a cartridge containing a factor or a nucleic acid encoding a factor for enhancing wound healing. The user may further select a cartridge/bioactive agent based on the location of the wound as different agents may be more effective for wounds at particular tissue sites.

The bioactive agent administered to the tissue site may be a nucleic acid and may optionally encode a growth factor such as, but not limited to activin (e.g., GI: 33563042, accession AB116641.1), amphiregulin (e.g., GI:179040, accession AAA51781.1), angiopoietins 1-4 (e.g., GI:20532340, accession NP_{—}001137.2), BMPs (e.g., GI:6680796, accession NP_{—}031580.1), betacellulin (e.g., GI:4502461, accession NP_{—}001720.1), CNNs (e.g., GI:2570234, accession AAB82065.1), cCAF, CTGF (Connective Tissue Growth Factor, e.g., GI: 4503123, Accession NM_{—}001901.1), CXC (e.g., GI:17385429, accession CAC83075.1), CXCR3 (e.g., GI:4504099, accession NP_{—}001495.1), Chemokins, cyr61 (e.g., GI:4996121, accession BAA78339.1), EGF (Epidermal Growth Factor, e.g., GI: 6978797, accession NM_{—}012842.1), eNOS (e.g., GI:40254422, accession NP_{—}000594.2), epigen (e.g., GI:60097950, accession NP_{—}001012404.1), epiregulin (e.g., GI:6679683, accession NP_{—}031976.1), FGF1-10 (Fibroblast Growth Factors 1-10, e.g., GI:41352694, accession NM_{—}002006), FSP-1 (e.g., GI:685015, accession AAB31954.1), FGFR (e.g., GI:54399701, accession AAV34170.1), Follistatin, GRO-α (e.g., GI:30023529, accession AAP13103.1), GM-CSF (Granulocyte-Macrophage Colony-Stimulating Factor, e.g., GI: 6983760, accession X55991.1), HGF (hepatocyte growth factor, e.g., GI:632774, accession AAB31855.1), HGFL (e.g., GI:42558273, accession NP_{—}835362.2), HGH (Human Growth Hormone, e.g., GI: 208528, accession K02382.1), HIF1-α (Hypoxia-Inducible Factor, e.g., GI: 31077211, accession NP_{—}851397), IGF-1 (Insulin-Like Growth Factor, e.g., GI: 34576544, accession NM_{—}184052), IL-1α (Interleukin-1α, e.g., GI: 47059075, accession NM_{—}010554.3), IL-1β (Interleukin-1β, e.g., GI:10835145, Accession NM_{—}000576.2), IL-10 (e.g., GI:49457009, accession CAG46825.1), iNOS (e.g., GI:9490415, accession AAB31028.2), inhibitins, Interferons, KGF (Keratinocyte Growth Factor, e.g., GI:15147344, accession NM-002009), Leptin (e.g., GI:6678678, accession NP_{—}032519.1), Lymphokins, MCP-1/CCL2 (e.g., GI:56745045, accession AAW28778.1), MIP2 (e.g., GI:66472262, accession NP_{—}001018356.1), MMP (e.g., GI:11342666, accession NP_{—}004521.1), MSP (e.g., GI:2196926, accession AAC83171.1), Mullerian Inhibiting substance (e.g., GI:248897, accession AAB22104.1), NDF1-4 (Neu differentiation factor, e.g., GI:248073, accession AAB21912.1), NGF (nerve growth factor, e.g., GI:263157, accession AAA03282.1), NO, neuregulin (e.g., GI:2459765, accession AAB71812.1), Nodals (e.g., GI:7305317, accession NP_{—}038639.1), nov (e.g., GI:1226019, accession CAA65404.1), PDGF (Platelet-Derived Growth Factor, e.g., GI: 34328144, accession NM_{—}008808), PLGF (e.g., GI:1666287, accession CAA70463.1), PRGF (e.g., GI:57489105, accession AAW51312.1), SF, Smad1-4 (e.g., GI:60414856, accession Q8BUN5), STAT1-3 (e.g., GI:6636498, accession AAF20200.1), TCRγ (e.g., GI:1396, accession CAA78309.1), TGF-α (Transforming Growth Factor-α, e.g., GI:311325, accession X71904.1), TGF-β (Transforming Growth Factor-β, e.g., GI: 63025222, accession NM_{—}000660.3), TIMP (e.g., GI:490094, accession CAA00898.1), TNF-α (Tumor Necrosis Factor, e.g., GI:57618953, accession: NM_{—}001009835.1), TNFRp55 (e.g., GI:30851242, accession AAH52675.1), t-PA (e.g., GI:14702169, accession NP_{—}127509.1), VEGF (Vascular Endothelial Growth Factor, e.g., GI: 76781486, accession NM_{—}001033756), WISP1-3 (e.g., GI:50926796, accession AAH78787.1), or combinations thereof. The nucleic acid may be in a dry or wet form. One particular dry form which provides long term stability is the lyophilized form. A nucleic acid thus administered to the tissue site may be transfected into cells at the tissue site and may be expressed in the cells to produce a desired factor or substance.

The cartridge further includes a second chamber which contains a liquid. Any liquid may be contained in the second chamber such as, but not limited to water, saline, ethanol, a buffer solution, a solution of PBS or other sterile diluent, an acid, an alkali, etc. In one example, the second chamber is adjacent to the first chamber and separated from the first chamber by a membrane. The membrane may include any separating element such as, for example, a wall, polymer, glass, plastic, polystyrene, silicone, rubber, Gortex, etc. Also, any arrangement of the first and second chamber is encompassed in the present invention. For example, the second chamber may be beneath, under, on top of, or over the first chamber. The second chamber may also be laterally oriented with respect to the first chamber. In one example, the second chamber is located at a distal end of the cartridge and the first chamber is located at a proximal end of the cartridge. In another example, the second chamber is located at a proximal end of the cartridge and the first chamber is located at a distal end of the cartridge. Alternatively, the second chamber may be either adjacent to the first chamber or separated from the first chamber by a predetermined distance. The first chamber and the second chamber may be separated by a distance of 0.05-1.0 cm. The first chamber and the second chamber may be separated by less than 0.05 cm, for example, 0.01 cm, 0.02, cm, 0.03 cm, or 0.04 cm. The first chamber and the second chamber may be separated by more than 1.0 cm (e.g., 1.1 cm, 1.2, cm, 1.4 cm, 1.5 cm, 2.0 cm, etc.).

A membrane separating the first chamber from the second chamber may be modified to result in mixing of the contents of the first chamber with the contents of the second chamber. Further, the cartridge may be shaken or otherwise agitated to facilitate mixing of the contents. Modification of the membrane may include, for example, rupture, dislocation or change in permeability of the membrane. The modification may cause the liquid to combine with the bioactive agent. Other potential modifications of the separating member include change in size, change in length, change in width, change in thickness, change in elasticity, change in tensile strength, change in configuration, change in shape, change in orientation, change in character, etc. Combination of the liquid with the bioactive agent may cause re-suspension, solubilization, or emulsion of the bioactive agent.

The bioactive agent may be administered at any desired dose or concentration.

For example, the bioactive agent may administered in a composition at a concentration ranging from 0.3-3.0 μg/μl (e.g., 0.3, 0.4, 0.5, 0.6, 0.7, 0.8, 0.9, 1.0, 1.2, 1.5, 1.7, 2.0, 2.2, 2.4, or 2.5 μg/μl). Larger or smaller doses or concentrations may be selected based on the condition being treated and/or the condition of the tissue site.

Optionally, an anaesthetic may be administered to the tissue site from the cartridge. The bioactive agent may be the anaesthetic. Alternatively, the bioactive agent may be administered with the anaesthetic, the anaesthetic being mixed with the bioactive agent and/or the liquid in the cartridge to form the composition to be administered to the tissue site. In one example, the anaesthetic is previously mixed with the bioactive agent and/or the liquid in the cartridge prior to mixing of the bioactive agent with the liquid. In another example, the anaesthetic is contained in a third chamber of the cartridge and a modification of a membrane separating the third chamber with either the first or the second chamber may cause mixing of the anaesthetic (from the third chamber) with either the bioactive agent or the liquid. The modification of the membrane separating the third chamber with either the first or the second chamber may undergo a similar modification as that described for the membrane separating the first chamber from the second chamber.

The cartridge may further include an orifice through which a composition may be delivered to a tissue site. For example, the cartridge may include a distal end in which the orifice is located. The orifice may be an opening in the cartridge and may be of any shape including round, ovoid, square, rectangular, triangular, irregularly shaped, etc. The orifice may also be within a needle or injection needle. The injection needle may be of any size such as, for example, 20 gauge-30 gauge. For example, the injection needle may be 20 gauge, 22 gauge, 24 gauge, 26 gauge, 28 gauge, or 30 gauge. In another example, the injection needle is larger than 20 gauge (e.g., 18 gauge, 14 gauge, 12 gauge, 10 gauge, etc). In another example, the injection needle is smaller than 30 gauge (e.g., 32 gauge, 35 gauge, etc).

The injection needle may extend from the cartridge at a predetermined length. For example, the injection needle may extend a predetermined length for intradermal administration of the bioactive agent. In another example, the injection needle extends from the cartridge a distance of 2-5 mm (e.g., 2.5 mm, 3.0 mm, 3.5 mm, 4.0 mm, 4.5 mm, etc.). In another example, the injection needle extends from the cartridge at a distance less than 2 mm (e.g., 0.5 mm, 1.0 mm, 1.5 mm).

Alternatively, the orifice may be within a tube, passageway, capillary tube, catheter, catheter tip, etc. The tube, passageway, capillary tube, catheter, catheter tip, etc., may be of any size and may extend from the cartridge at any length. The bioactive agent may be suspended in the liquid in the cartridge to form the composition which is delivered to the tissue site via the orifice.

The cartridge may further include a conductive portion which can be operatively connected to the at least one electrode at the distal end of the cartridge. The conductive portion may include, for example, electrical wires for conducting electrical pulses through the cartridge. Electrical pulses may be received at a proximal end of the cartridge and conducted or transmitted via the conductive portion to the distal end of the cartridge. The electrical pulses are administered to the tissue site via at least one electrode at the distal end, in this embodiment.

The cartridge may further include at least one electrode for administering an electrical field to the tissue site. In one example, the at least one electrode or a plurality of electrodes is located at a distal end and extends a distance from the distal end. The distance may be any length for application of an electrical pulse. For example, the at least one electrode or the plurality of electrodes may extend from the cartridge 0.5 mm-4.5 mm (e.g., 0.6, 0.7, 0.9, 1.0, 1.2, 1.5, 1.7, 1.9, 2.0, 2.5, 3.0, 3.5, 4.0, or 4.4 mm). The at least one electrode or the plurality of electrodes may be arranged circumferentially around the injection needle. The at least one electrode or the plurality of electrode needles may be longer than the injection needle containing the orifice, shorter than the injection needle containing the orifice, or the same length as the injection needle containing the orifice. If each of the electrodes is shorter than the injection needle containing the orifice, then the electrodes may extend into the tissue site to a lesser degree than the injection needle or may not extend into the target tissue site at all.

The at least one electrode may be located on a common facet of the cartridge as the orifice such that a composition or bioactive agent may be administered to a tissue site via the orifice and an electric field may be administered to the tissue site via the at least one electrode. The at least one electrode may be located circumferentially around the orifice.

Alternatively, the cartridge may not itself include the at least one electrode or the plurality of electrodes but may be attachable to at least one electrode or plurality of electrodes. For example, the at least one electrode may snap on, screw on, or otherwise attach to a distal end of the cartridge.

An apparatus for administering or delivering a bioactive agent to a tissue site on a subject comprises the cartridge. The cartridge may be detachable, disposable, and/or autoclavable. The apparatus may be hand-held and may be battery-operated. The battery may be rechargeable or may be non-rechargeable. Alternatively, the apparatus may be a wall unit. The apparatus may also be powered by AC or DC current via a wall plug and electrical outlet.

The apparatus includes a means for combining the bioactive agent and the liquid to form a composition. For example, the cartridge may be attached to the means for combining the bioactive agent and the liquid. In one example, the means for combining the bioactive agent and the liquid includes a plunger. Alternatively, the means for combining the bioactive agent may be a button, latch, lever, switch, etc. The cartridge may be snapped onto, screwed onto, or otherwise attached to the means for combining the bioactive agent and the liquid. The attachment of the cartridge to the means for combining the bioactive agent and the liquid may optionally cause mixing of the bioactive agent and the liquid within the cartridge. In another example, the means for combining the bioactive agent and the liquid may provide mechanical energy to the cartridge for causing the combination or mixing of the bioactive agent and the liquid.

The means for combining the bioactive agent may include a plunger that may contact the cartridge. For example, the plunger may contact a proximal end of the cartridge by an operator. Contact of the plunger with the cartridge may cause a modification of a membrane separating the bioactive agent and the liquid. The modification of the membrane may include, for example, rupture, dislocation, change in permeability, change in size, change in length, change in width, change in thickness, change in elasticity, change in tensile strength, change in configuration, change in shape, change in orientation, change in character, etc. The modification may result in the mixing of the bioactive agent and the liquid to form the composition in the cartridge. Optionally, shaking or vibrating the apparatus may facilitate mixing of the contents of the first and second chambers.

The composition may be expelled from the cartridge through the orifice under pressure. The pressure may be caused by the means for combining the bioactive agent and the liquid which may include a mechanism for providing energy to the cartridge, the energy causing an increased pressure within the cartridge. The increased pressure within the cartridge may cause the contents of the cartridge (e.g., the suspended bioactive agent) to pass through the orifice to the tissue site. The cartridge may be disposable and may be detached from the gun applicator device after use and discarded. The apparatus may be used to administer a nucleic acid to a tissue site. Any bioactive agent may be delivered to a tissue site in need of the bioactive agent.

The apparatus includes at least one electrode for administering an electric field to the tissue site. The electric field may cause opening of pores in membranes of cells at the tissue site such that a composition administered at the tissue site may enter the cells across the opened pores. The pores may be transitory or longer lasting. The electric field may be applied after the composition is administered to enhance effectiveness of the composition at the tissue site. For example, the electric field may be applied immediately after the composition is administered. In one example, the electric field applied immediately after the composition is administered is applied less than one second after the composition is administered. In another example, the electric field applied immediately after the composition is administered is applied 1-15 seconds after the composition is administered. Alternatively, the electric field may be applied prior to administration of the composition (e.g., 1-15 seconds before administration of the composition. In another example, the electric field may be applied at the same time as the administration of the composition. In yet another example, the electric field is applied a predetermined time period after the administration of the composition (e.g., greater than 15 seconds after administration of the composition).

The apparatus may comprise preset controls corresponding to particular tissue sites. The controls may determine pulse strength, duration, frequency, or number of pulses. For example, if an electric field is to be applied on the skin, colon, intestines, liver, spleen, or any other organ or tissue, the electroporation parameter settings may be selected according to values optimized for the type of tissue.

Many different types of electric pulses may be applied in the electroporation of a tissue site. For example, the electric pulse may be in the form of square wave pulses, exponential waves, unipolar oscillating wave forms, bipolar oscillating wave forms, other wave forms generating electric fields, or a combination of any of these forms. In one example, a square wave pulse is applied to wound tissue of a subject. The electroporation parameters of the applied electric pulse (e.g., volts, duration, pulse interval, number of pulses, etc) may be adjusted according to the type of tissue involved. In one example, the electroporation parameters include 1-30 pulses at an interval of 10-200 ms, voltage of 5 to 1800 volts, and/or a duration of 1 μsec to 800 μsec. In another example, a train of 6-18 pulses is used to electroporate mammalian tissues. In another example, the pulses are at a duration of 10 μsec to 800 μsec.

The apparatus may include a safety feature such as a fuse, a circuit breaker, a safety switch, etc. to prevent excess field strength from being applied to the tissue site. The electroporation parameters may be controlled such that when the parameters are set to values that result in an electric field that is excessive for the designated tissue site, the safety feature disables the apparatus from applying the electric field. In another example, the safety feature does not disable the apparatus but allows application of an electric field that is less than the electric field corresponding to the electroporation parameters. In this case, the electric field applied may be at a setting that is the maximum setting permissible for the particular tissue site.

A wound at a tissue site may be treated with an apparatus according to the present invention by delivery of a macromolecule to the wound at the tissue site. Any wound may be treated with the apparatus including but not limited to burn wounds, surgical wounds, pressure wounds, traumatic wounds, etc. The apparatus may deliver nucleic acid molecules coding for a compound useful in the treatment of the wound. For example, a nucleic acid encoding growth factors or cytokines may be administered to a tissue site. The term “wound” refers to any disruption of the organ structure due to an outside stress such as a physical, chemical or biological agent. This may include, but is not limited to, any disruption created by trauma, thermal burn, radiation, infectious agent, or chemical agent. Wounds located at any location in the body may be treated and include wounds of all types including wounds caused by trauma, surgery, any medical therapy, or dermatological conditions including cosmetic treatments. The bioactive agent may be applied on any part of the skin of the body as well as on wounds in internal organs such as but not limited to muscle, stomach, esophagus, small bowel, colon, spleen, liver, salivary gland, bone, tendon, nerves and bladder. Tumors can also be targeted.

In the case of wound healing at a tissue site, the apparatus may generate and apply at least one electric pulse to a skin surface, wound surface or to a surface adjacent or in proximity to a wound surface at sufficient strength and duration to open pores in the cell membranes of cells in or around the wound. Thus a composition or bioactive agent administered as described may gain entry into the cytoplasm and/or nucleus of cells at a tissue site via the open pores.

A nucleic acid (e.g., the plasmid encoding for a growth factor) may traverse the cytoplasm of the cell, cross the nuclear membrane of the cell and may enter the nucleus where the nucleic acid may be inserted into the nuclear DNA. The nucleic acid encoding the growth factor may be transcribed and translated to produce the encoded growth factors. The increased production of growth factors results in improved healing.

Only one regimen of treatment may be necessary. The apparatus further obviates the need for frequent or repeated applications of compounds, factors or growth factors. Moreover, the device is easy to use, making use of the device in an out-patient setting just as convenient as in the operating room during surgery or at an in-patient's bedside. In addition, in the example of wound healing, the device does not rely on the application of creams or lotions whose absorption is less than ideal due to potential blockage of absorption by the typical scab or eschar present on the surface of wounds. The apparatus may allow for injection of a therapeutic agent through the eschar.

As described above, a composition may be administered to a wound, the composition containing a plasmid construct of a cDNA coding for a growth factor cloned into an expression vector using a promoter. The promoter may be but is not limited to a CMV promoter. Many growth factors may be used according to the present invention. Examples of growth factors that may be administered to a wound according to one aspect of the present invention include, but are not limited to, Keratinocyte Growth Factor (KGF), Fibroblast Growth Factors 1-10 (FGF1-10), Vascular Endothelial Growth Factor (VEGF), Platelet-Derived Growth Factor (PDGF), Insulin-Like Growth Factor (IGF-1), Hypoxia-Inducible Factor (HIF1-α), Transforming Growth Factor-α (TGF-α), Transforming Growth Factor-β (TGF-β), Connective Tissue Growth Factor (CTGF), Interleukin-1α (IL-1α), Interleukin-1β (IL-1β), Human Growth Hormone (HGH), Granulocyte-Macrophage Colony-Stimulating Factor (GM-CSF), Epidermal Growth Factor (EGF), Activin, and/or Tumor Necrosis Factor (TNF-α), to name a few. Also, the composition may include other macromolecules in addition to or instead of growth factors such as, but not limited to, proteins, cytokines, liposomes, other nucleic acids or any other molecule with a demonstrated biological effect or any combination thereof. By varying the components of the composition to include components for enhancing various aspects of wound healing, a user may select the desired components in the administered composition based on the type, location or other characteristics of the wound to be healed.

The composition may be administered locally to the site of the wound or to areas juxtaposed to, adjacent to, or in proximity to the wound. In one example, the wound is a cutaneous wound and the composition may be delivered intradermally to the wound. In this example, the device may include a needle for injecting the composition intradermally into the wound or into intradermal areas surrounding the wound for improved wound healing.

The concentration of the compound used in the composition may vary based on many factors such as size of the wound or size/weight of the subject being treated. For example, in our experience with laboratory animals such as mice and rats, the amount of DNA compound used to successfully treat 5 mm diameter wounds has ranged from 40 to 100 μg of DNA plasmid diluted into 40 to 100 μl of PBS. (Electroporative transfection with KGF-1 DNA improves wound healing in a diabetic mouse model. Marti G, Ferguson M, Wang J, Byrnes C, Dieb R, Qaiser R, Bonde P, Duncan M D, Harmon J W. Gene Ther. 2004 December; 11(24):1780-5). Other dosages, including larger concentrations, may be selected to enhance wound healing while avoiding toxicity or adverse systemic effects. For example, a 10 cm diameter ulcer in a human being may need multiple injections. In one example, 12 repeated injections around the edges of the wound totaling 480 to 1,200 μg of DNA in a 480 to 1,200 μl solution of PBS or other sterile diluent may be administered. Examples of a sterile diluent that may be used in the present invention include but are not limited to Sodium Chloride, water, ethanol.

In addition, an electrical field (i.e., an electric pulse may be administered to the wound) may be applied to the wound either before, during or after administration of the composition. In one example, the electric field may be applied immediately after administration of the composition (e.g., up to 10-30 seconds after administration of the composition). Thus the apparatus may provide both administration of a composition or compound for improved wound healing and electroporation. The electric pulse administered to the wound area, as describe above, may cause enhanced uptake of macromolecules for wound healing.

### EXAMPLE 1

### Apparatus

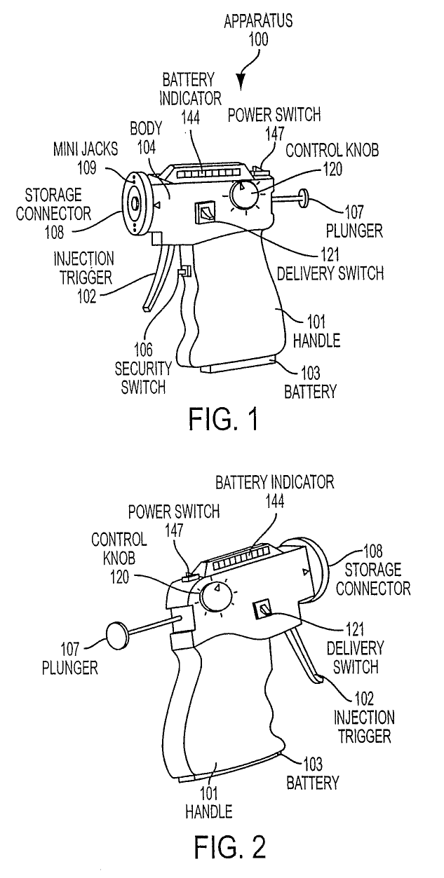

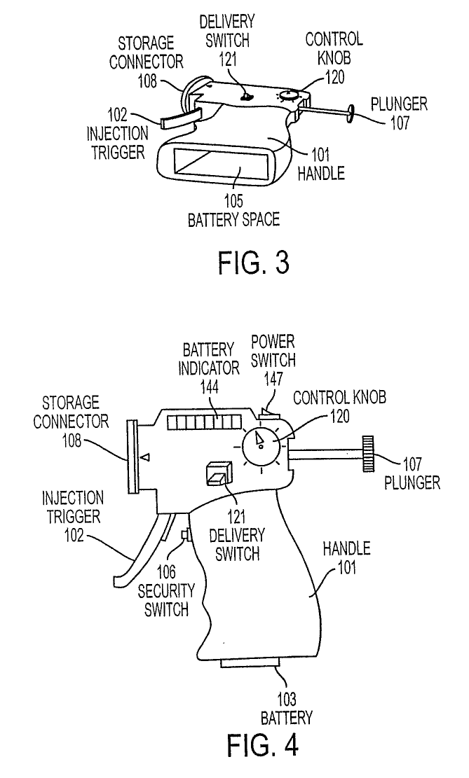

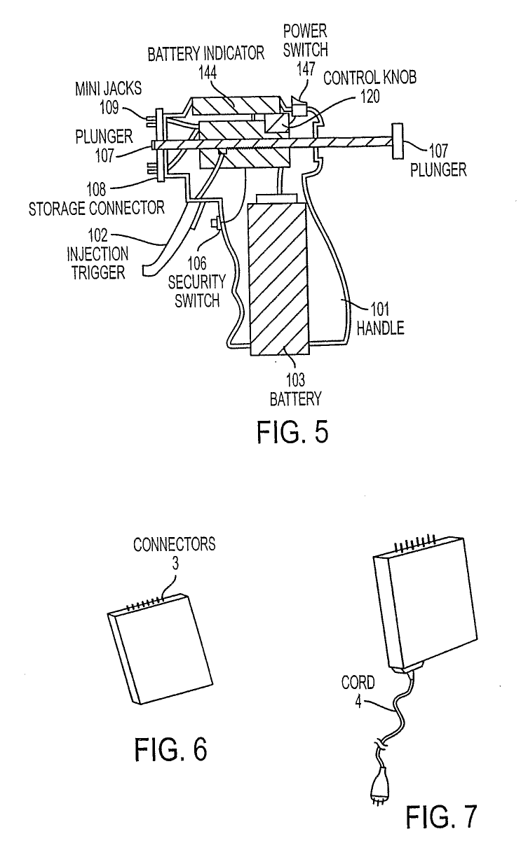

FIGS. 1-5 illustrate an example of a device or apparatus according to one aspect of the present invention. In this example, the apparatus is used to administer a bioactive agent for enhancing wound healing. FIG. 1 is a side/frontal perspective view of the device. FIG. 2 is a side/rear perspective view of the device. FIG. 3 is a bottom perspective view of the device. FIG. 4 is a side elevation view of the device. FIG. 5 is a cross sectional side view of the device. In this example, the apparatus 100 contains a body 104 and a handle 101 attached to the body 104. The handle may further contain a hollow cavity or battery space 105 in which a battery 103 may be housed. FIG. 6 is a side/top perspective view of an example of a battery of the apparatus. The battery 103 may charge the apparatus and may also be rechargeable. Thus, in this example, a battery 103 may be inserted into a hollow cavity in the handle 101 of the apparatus 100 and may connect to the apparatus 100 via connectors 180 to allow transmission of electric current upon activation of the apparatus 100. In another example, the battery 103 may include a cord 181 that may plug into an AC power source. FIG. 7 is a side/top perspective view of an example of a battery that is plugged into an AC power source.

The handle 101 is operatively connected to a body 104 of the apparatus 100. The body 104 of the apparatus contains an injection trigger 102 for activation of the apparatus 100, a plunger 107, a storage connector 108, and/or mini-jacks 109 as described in the examples herein. In addition, a cartridge, such as a DNA storage bicameral cartridge, may be connected to the apparatus 100 via the storage connector 108, for example.

The injection trigger 102 may be depressed or activated by a user. Activation of the injection trigger 102 may cause the plunger 107 to advance through the body 104 of the apparatus 100. For example, as a user grasps the handle 101 of the apparatus 100 and presses the injection trigger 102 toward the handle 101, the movement of the injection trigger 102 causes the plunger 107 to incrementally slide into the body 104 of the apparatus 100. As the example of FIGS. 1-5 illustrate, the plunger 107 advances from a rear side of the body 104 toward a front side of the body 104. As the plunger 107 advances into the body 104 of the apparatus 100, an end of the plunger 107 may be advanced to the front side of the body 104. As seen in the example of FIG. 1, a front tip of the plunger 107 may contact the front side of the body 104 where a cartridge may be attached.

Advancement of the plunger 107 via the injection trigger 102 may be accomplished in a variety of ways. As one non-limiting example, the plunger 107 may be a rod-shaped member containing ridges or inward or outward indentations such that movement of the injection trigger 102 that engages with the plunger 107 may cause a ratcheting-type advancement of the plunger 107 through the body 104 of the apparatus 100.

In this example, the front tip of the plunger 107 may advance to the storage connector 108 at the front side of the body 104 where a cartridge may be connected to the body 104. The storage connector 108 may contain mini jacks 109 for connecting the cartridge to the apparatus 100. In one example, the storage connector 108 may snap onto the apparatus to connect the cartridge with the apparatus 100. The connection may be further secured by various means. For example, the connection may be secured by a clip, a mounting bracket, a threaded ring, or a locking dent. Also markings on the apparatus, storage connector 108 and/or the cartridge may provide a visual cue to a user for alignment of the components for a proper connection.

Contact with the storage connector 108 and the cartridge at the front of the body 104 may result in activation of the cartridge, administration of a composition stored in the cartridge and administration of at least one electric pulse to enhance wound healing as described herein.

The apparatus 100 may supply an electric pulse or a plurality of electric pulses. Characteristics of the electric pulse may be controlled by the user. For example, the apparatus 100 may contain a control knob 120 for setting electroporation parameters corresponding to a desired electric pulse. For example, a voltage level, pulse duration, pulse interval, a number of pulses, etc. may be adjusted by a user via the control knob 120. In another example, electroporation parameter settings may be preset based on type of tissue to be treated. In this example, the control knob 120 may be set to a setting based on the location of the wound to be treated such as, but no limited to, skin, muscle, liver, spleen, small intestine, colon, stomach, esophagus, bladder, etc. In yet another example, the apparatus 100 may contain a microchip (not shown) for storing electroporation parameter settings. A user may apply the desired electroporation via the control knob 120 which may cause the apparatus to access the electroporation parameter settings stored on the microchip. Also, the control knob 120 may be secured to avoid accidental or undesired resetting of the electroporation parameters. The microchip may further store information pertaining to previously applied electroporation parameters.

The apparatus 100 may further include a delivery switch 121 for administration of an electric pulse. For example, the apparatus 100 and attached cartridge may be placed in proximity to a wound. The apparatus 100 may be activated by depressing the injection trigger 102 to cause a plunger 107 to advance incrementally through the body 104 of the apparatus 100. A front tip of the plunger 107 may advance from a rear side of the body 104 to a front side of the body 104 to contact a cartridge attached to the front side of the body 104 via a storage connector 108. A compound (e.g., cDNA encoding for growth factors) may thus be released at the wound site. A user may control electroporation parameters via the control knob 120 and may administer electric pulses according to the electroporation parameters selected to the wound site by depressing the delivery switch 121.

The apparatus may also include any number of safety features to prevent the application of electric pulses of excessive magnitude, duration or frequency. For example, the parameters may be controlled or the apparatus may further contain fuses, circuit breakers or switches for controlling the administration of electric pulses.

The apparatus 100 may further include an optional security switch 106 for regulating the application of current via the apparatus 100. For example, if the security switch 106 is depressed, current delivery from the battery 103 of the apparatus 100 is enabled. However, if the security switch is not depressed (i.e., in an inactive state), then current delivery is disabled such that current may not be provided to the apparatus 100 from the battery 103. In one example, a composition or compound for enhancing wound healing is administered via the apparatus 100 and an electrical field is applied to the affected area after the composition or compound is administered. For example, the security switch 106 may be depressed only after the injection trigger 102 or the plunger 107 has advanced a predetermined distance. Thus, delivery of an electric pulse will be enabled only after the compound/composition has been delivered to the wound area, in this example.

### EXAMPLE 2

### Cartridge

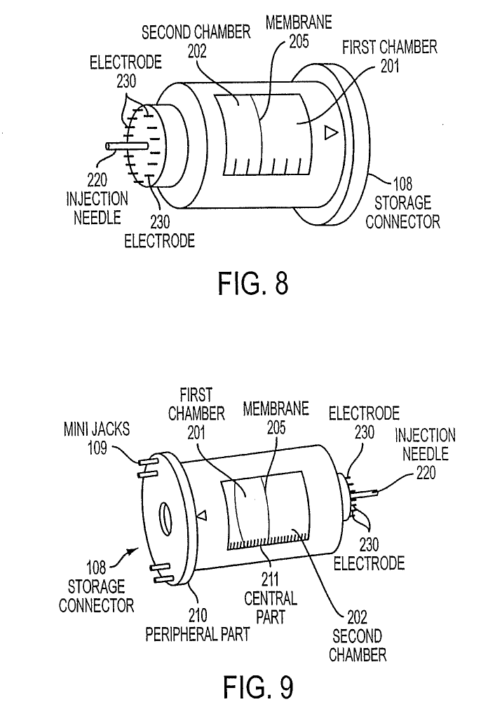

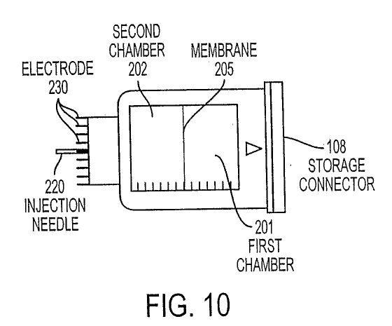

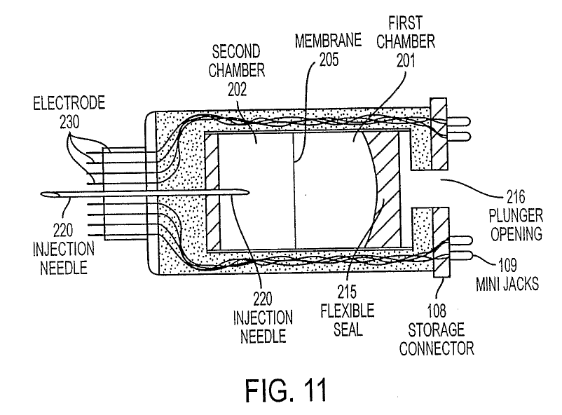

As seen in the different views of an example of a cartridge of FIGS. 8-11, the cartridge 200 may contain at least one chamber for storing a composition. FIG. 8 is a side/frontal perspective view, FIG. 9 is a side/rear perspective view, FIG. 10 is a side elevation view, and FIG. 11 is a cross sectional side view. The composition stored in the cartridge in this example may contain a composition for enhancing wound healing when applied to a wound area. In the example of FIGS. 8-11, the cartridge 200 is a bicameral cartridge including a first chamber 201 and a second chamber 202 and an intervening membrane 205 between the first chamber 201 and the second chamber 202. The first chamber 201 may contain a composition of cDNA in a precipitated form and the second chamber 202 may contain a buffer solution for suspension of the cDNA prior to injection. In one example, the cartridge 200 is connected to the body 104 of the apparatus 100 via a storage connector 108 via mini jacks 109. Upon connection of the cartridge 200 to the apparatus 100, the buffer solution in the second chamber 202 is capable of being released from the second chamber 202 into the first chamber 201 with the stored cDNA therein to resuspend the cDNA prior to injection to the wound site.

The cartridge 200 may further contain at least one injection needle 204 and at least one electrode 203. The re-suspended cDNA in this example may be administered to a wound site via the at least one injection needle 204 through a pressure-injection system, for example. Also, at least one electric pulse may be administered to the wound site via the at least one electrode.

FIGS. 8-11 illustrate different views of one example of a cartridge of the present invention. In this example, the cartridge has a peripheral part 210 and a central part 211. The peripheral part 210 may contain an electrical wiring system for conducting an electric pulse. For example, an electrical pulse originating from the apparatus 100 may be transmitted through the peripheral part 210 of the cartridge and may further be applied to a subject by at least one electrode connected to the electrical wiring system in the peripheral part 210 of the cartridge.

The central part 211 may contain at least one chamber containing a compound for administration to a subject in need of the compound. The central part 211 of the cartridge contains two chambers (i.e., is a bicameral cartridge), i.e., a first chamber 201 and a second chamber 202, separated by a membrane 205.

In addition, the cartridge may further contain a flexible seal 215 at a proximal end of the first chamber 201 which may separate the first chamber 201 from the tip of the plunger 107. In this example, the plunger 107 may enter the cartridge from the apparatus 100 through a plunger opening 216. After extending through the plunger opening 216, the plunger 107 may contact the flexible seal 215. As the plunger 107 continues to advance into the plunger opening 216, the flexible seal 215 is re-shaped to decrease the volume of the first chamber 201, thus causing an increase of pressure within the first chamber 201. The increase pressure in the first chamber 201, in turn, may cause the membrane 205 to rupture so that the contents of the first chamber 201 may be mixed with the contents of the second chamber 202. Alternatively, the increase pressure may cause the membrane 205 to become dislocated from an original position (rather than rupturing) to allow the diluent in the first chamber 201 to enter the second chamber 202.

In this example, the second chamber 202 may contain DNA for administration to a subject. The DNA contained within the second chamber 202 may be dry DNA so that freezing of the DNA is not necessary. Drying of DNA may be accomplished in a variety of ways. For example, the DNA may be freeze dried or may be dried through evaporation of a volatile solvent.

By storing dry DNA in the second chamber 202, the dry DNA may be easily stored, for example, at room temperature (rather than at −80° C.) and has a long shelf life. The first chamber 201 may contain a diluent solution for suspending the DNA in solution prior to administration to a subject. Thus, when the plunger 107 causes deformation of the flexible seal 215 and rupture of the membrane 205, the diluent solution in the first chamber 201 may mix with dry DNA in the second chamber 202 to cause suspension of the dry DNA in solution so that the DNA may be properly administered to a subject. Further pressure exerted via the plunger 107 or repeated pressure from the plunger 107 may cause increased pressure within the chambers of the cartridge which may cause the DNA suspended in solution through an injection needle 220 for administration to a subject (i.e., a wound site).

In another example, the walls of the cartridge may be transparent to allow the user to observe the dilution or mixing within the chambers of the cartridge. After delivery of the substance and completion of therapy, the cartridge may be removed from the apparatus and discarded.

### EXAMPLE 3

### The Electrode System

In addition, the cartridge may contain an electrode system for applying an electric pulse to a wound of a subject. As FIGS. 8-11 illustrate, the cartridge may contain at least one electrode “needle”. In this example, a plurality of electrodes are disposed circumferentially at a distal end of the cartridge around an injection needle 220. The electrodes may be of any desired shape, length or type and may be spaced at distances based on the surface over which the electric pulse is to be applied. For example, if the electrodes are spaced close together, the electric field generated may be more limited so that the electric field may be applied to a specific desired region (e.g., a wound site). In one example, the electrodes are shorter than the length of the injection needle 220. In another example, the electrodes are longer than the injection needle 220. In another example, the electrodes are the same length as the injection needle 220.

The electrodes are circumferentially arranged at a distal end around a central injection needle of the cartridge and are connected to an electric wire system running through the peripheral portion of the cartridge. For example, the electrode needles may be spaced approximately 1 mm apart. The electrode needles may be used to apply an electric pulse to a wound area and the injection needle may be used for applying a compound or composition to the wound area to improve healing of the wound. In another example, the electrode needles are 30 gauge needles or less and the injection needle is 20-30 gauge.

In another example, the electrode needles and the injection needle may be separated by a non-conductive material such as plastics or polymer resins as well as hypo-allergenic or non-allergenic material.

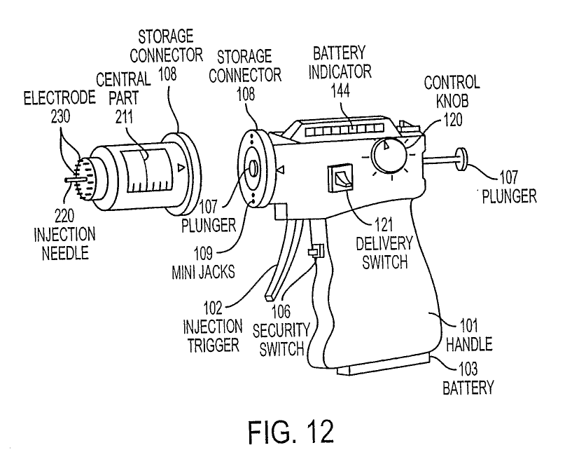

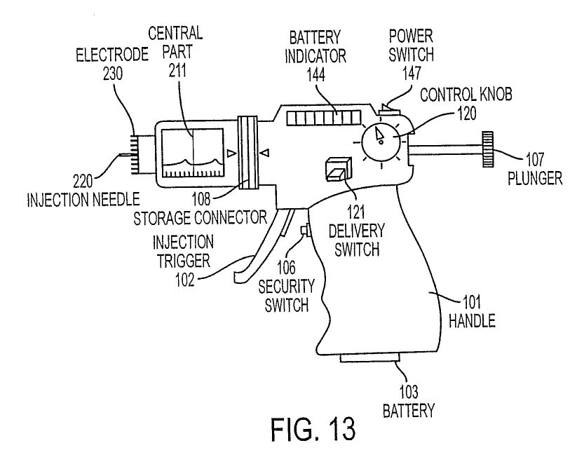

FIGS. 12 and 13 illustrate an example of an apparatus and cartridge assembly for delivery of a bioactive agent to a tissue site. As FIGS. 12 and 13 illustrate, the cartridge may be attached in the apparatus. The cartridge contains a pre-stored bioactive agent and/or a liquid for suspending the pre-stored bioactive agent. The cartridge may be snapped onto, screwed onto or otherwise attached in the apparatus to a means for combining the bioactive agent and the liquid (illustrated as the body 104 and handle 103 in this example). Once attached, mixing of components of the composition for delivery to the tissue site (e.g., combination or mixing of the bioactive agent with a liquid to form the composition) occurs. The means for combining the bioactive agent and the liquid may provide sufficient energy to cause mixing of the bioactive agent with the liquid and/or expulsion of the suspended bioactive agent to the tissue site.

### EXAMPLE 4

### Wound Healing in a Rat Model

We have previously found delayed or impaired wound healing in a rat septic model. Animals underwent partial cecal ligation via laparotomy and then four standard cutaneous 8 mm punch biopsies were performed on the dorsal skin. Transfection was carried out with DNA plasmid expression vectors for luciferase and KGF with and without electroporation. Plasmid injection and electroporation was performed at time of wounding (Day 0). Wound surface area was scanned and measured over time and luciferase activity quantified photometrically.

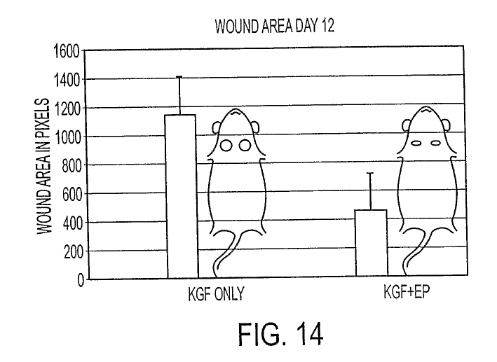

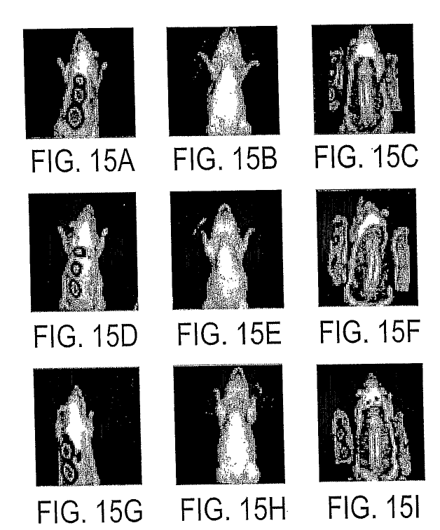

Electroporation enhanced luciferase expression four-fold by day 17 compared to plasmid without electroporation (28.2±9.8 vs. 7.0±0.8; p<0.02); and double electroporation increased expression over five-fold (75.5±32.6 vs. 13.1±8.0; p<0.02). Single electroporation transfection of a plasmid vector expressing keratinocyte growth factor (KGF) improved wound healing as evidenced by an average of 33.2% smaller wound areas at Day 12 in the treated compared to the untreated animals (2359±339 vs. 3533±367, average (pixels)±SEM). The Mann-Whitney Rank Sum test shows a statistically significant difference between the median value of the two groups (P=0.005). FIGS. 15A-15I illustrate transfection of a KGF plasmid vector to wound sites on a rat model. As seen in FIG. 14, after administration of KGF plasmid vector and electroporation to the wound sites, the rat exhibited improved wound healing as evidenced by an average of 60.0% smaller wound areas by Day 12 in the animals treated with KGF and electroporation as compared to animals treated only with KGF (1149+−260 vs. 460+−78 pixels, p<0.009).

FIGS. 15A-15I demonstrate that the effect of the treatment administered within cutaneous tissue with an intradermal injection as compared to a subcutaneous injection that did not show any luciferase expression. As FIG. 16 illustrates, intradermal injection of a luciferase plasmid to the left dorsum of each rat with mirror image subcutaneous injections on the right dorsum was performed. The subcutaneous injections did not result in any transfection as demonstrated by minimal luciferase signal.

The present invention includes any novel feature or combination of features disclosed herein either explicitly or any generalization thereof. While the invention has been described with respect to specific examples including presently preferred modes of carrying out the invention, those skilled in the art will appreciate that there are numerous variations and permutations of the above described systems and techniques. Thus, the spirit and scope of the invention should be construed broadly as set forth in the appended claims.

What is claimed is:

1. A cartridge for the delivery of a composition to a tissue of a subject, comprising:
a first chamber comprising a bioactive agent;
a second chamber comprising a liquid;
an orifice through which the composition is administered;
a conductive portion for transmitting an electrical pulse.

2. The cartridge of claim 1 further comprising at least one electrode operatively connected to the conductive portion.

3. The cartridge of claim 2 wherein the at least one electrode is a needle electrode.

4. The cartridge of claim 2 wherein the at least one electrode is arranged circumferentially around the orifice.

5. The cartridge of claim 2 wherein the orifice is in a needle, the needle extending further from the cartridge than the at least one electrode so that the needle can penetrate the tissue deeper than the at least one electrode.

6. The cartridge of claim 1 wherein the conductive portion comprises electrical wires.

7. The cartridge of claim 1 wherein the orifice is in a needle.

8. The cartridge of claim 1 wherein the bioactive agent comprises a macromolecule for enhancing healing of a wound in the subject.

9. The cartridge of claim 8 wherein the wound is a burn wound.

10. The cartridge of claim 1 wherein the bioactive agent comprises a chemotherapeutic agent.

11. The cartridge of claim 1 wherein the bioactive agent comprises a macromolecule for treating a skin disorder.

12. The cartridge of claim 11 wherein the skin disorder comprises a degenerative disorder.

13. The cartridge of claim 12 wherein the degenerative condition comprises alopecia.

14. The cartridge of claim 1 wherein the bioactive agent comprises a macromolecule for treating infections.

15. The cartridge of claim 1 wherein the macromolecule is a reporter macromolecule.

16. The cartridge of claim 1 wherein the bioactive agent comprises a nucleic acid.

17. The cartridge of claim 16 wherein the nucleic acid is lyophilized.

18. The cartridge of claim 16 wherein the nucleic acid is in a dry form.

19. The cartridge of claim 1 wherein the bioactive agent comprises a peptide.

20. The cartridge of claim 19 wherein the peptide comprises one of a growth hormone and a cytokine.

21. The cartridge of claim 1 wherein the bioactive agent is solubilized by the liquid to form the composition.

22. The cartridge of claim 1 further comprising a membrane separating the first chamber and the second chamber, wherein the membrane is modifiable to permit mixing of the bioactive agent and the liquid to form the composition.

23. The cartridge of claim 1 which is disposable.

24. The cartridge of claim 1 wherein the bioactive agent comprises a nucleic acid encoding a growth factor.

25. The cartridge of claim 24 wherein the growth factor is selected from the group consisting of Activin, amphiregulin, angiopoietins 1-4, BMPs, betacellulin, CNNs, cCAF, CTGF, CXC, CXCR3, Chemokins, cyr61, EGF, eNOS, epigen, epiregulin, FGF1-10, FSP-1, FGFR, Follistatin, GRO-α, GM-CSF, HGF, HGFL, HGH, HIF1-α, IGF-1, IL-1α, IL-1β, IL-10, iNOS, inhibitins, Interferons, KGF, Leptin, Lymphokins, MCP-1/CCL2, MIP2, MMP, MSP, Mullerian Inhibiting substance, NDF1-4, NGF, NO, neuregulin, Nodals, nov, PDGF, PLGF, PRGF, SF, Smad1-4, STAT1-3, TCRγ, TGF-α, TGF-β, TIMP, TNF-α, TNFRp55, t-PA, VEGF, WISP1-3, and combinations thereof.

26. A method for delivering a liquid composition to a tissue site in a subject comprising:
modifying a separating element between a bioactive agent and a liquid in separate chambers to cause solubilization of the bioactive agent by the liquid to form the liquid composition;
administering the liquid composition to the tissue site in the subject; and
establishing an electrical field at the tissue site in the subject.

27. The method of claim 26 wherein the bioactive agent comprises a nucleic acid encoding a growth factor, the nucleic acid being in a dry form.

28. The method of claim 26 wherein modifying the separating element comprises rupturing the separating element to permit mixing of the bioactive agent and the liquid.

29. The method of claim 26 wherein the step of administering the composition is performed immediately after the modifying step.

30. The method of claim 26 further comprising a step of shaking the liquid composition after the modifying step.

31. The method of claim 26 wherein step of establishing the electrical field is performed after the administering step.

32. The method of claim 26 wherein the step of administering the composition is performed simultaneously with the step of establishing.

33. The method of claim 26 wherein the tissue site contains a wound.

34. The method of claim 33 wherein the wound is a burn wound and wherein the liquid composition is for treating the burn wound.

35. The method of claim 26 wherein the tissue site is skin.

36. The method of claim 35 wherein the skin exhibits alopecia.

37. The method of claim 26 wherein the tissue site is a scar.

38. The method of claim 37 wherein the scar is a hypertrophic scar.

39. The method of claim 37 wherein the scar is an inflammatory scar.

40. The method of claim 26 wherein the tissue site is selected from the group consisting of muscle, stomach, esophagus, small bowel, colon, spleen, liver, salivary gland, bone tendon, nerves, and bladder.

41. The method of claim 26 wherein the bioactive agent comprises a nucleic acid encoding a growth factor.

42. The method of claim 41 wherein the growth factor is selected from the group consisting of Activin, amphiregulin, angiopoietins 1-4, BMPs, betacellulin, CNNs, cCAF, CTGF, CXC, CXCR3, Chemokins, cyr61, EGF, eNOS, epigen, epiregulin, FGF1-10, FSP-1, FGFR, Follistatin, GRO-α, GM-CSF, HGF, HGFL, HGH, HIF1-α, IGF-1, IL-1α, IL-1β, IL-10, iNOS, inhibitins, Interferons, KGF, Leptin, Lymphokins, MCP-1/CCL2, MIP2, MMP, MSP, Mullerian Inhibiting substance, NDF1-4, NGF, NO, neuregulin, Nodals, nov, PDGF, PLGF, PRGF, SF, Smad1-4, STAT1-3, TCRγ, TGF-α, TGF-β, TIMP, TNF-α, TNFRp55, t-PA, VEGF, WISP1-3, and combinations thereof.

43. An apparatus for the delivery of a composition to a tissue of a subject comprising:
a cartridge comprising:
a first chamber containing a bioactive agent;
a second chamber containing a liquid; and
at least one orifice;
an energy source for generating an electric field;
a means for combining the bioactive agent and the liquid to form a composition;
at least one electrode for delivering the electric field to the tissue.

44. The apparatus of claim 43 wherein the means for combining comprises a plunger.

45. The apparatus of claim 43 wherein the cartridge comprises a membrane separating the first chamber and the second chamber.

46. The apparatus of claim 45 wherein the means for combining causes a modification of the membrane.

47. The apparatus of claim 46 wherein the means for combining causes an increase in pressure in at least one of the first chamber and the second chamber.

48. The apparatus of claim 46 wherein the modification of the membrane comprises dislocation of the membrane.

49. The apparatus of claim 46 wherein the modification of the membrane comprises change of permeability of the membrane.

50. The apparatus of claim 46 wherein the modification comprises rupture of the membrane.

51. The apparatus of claim 43 wherein the bioactive agent comprises a macromolecule capable of enhancing healing of a wound in the subject.

52. The apparatus of claim 51 wherein the wound is a burn wound, and wherein the composition is for treating the burn wound.

53. The apparatus of claim 43 wherein the bioactive agent comprises a macromolecule for treating a skin disorder.

54. The apparatus of claim 53 wherein the skin disorder is a degenerative disorder.

55. The apparatus of claim 54 wherein the skin disorder comprises alopecia.

56. The apparatus of claim 43 wherein the bioactive agent comprises an antibiotic.

57. The apparatus of claim 43 wherein the bioactive agent comprises a hormone.

58. The apparatus of claim 43 wherein the bioactive agent comprises a tagged macromolecule.

59. The apparatus of claim 43 wherein the bioactive agent comprises a nucleic acid encoding a growth factor.

60. The apparatus of claim 59 wherein the growth factor is selected from the group consisting of Activin, amphiregulin, angiopoietins 1-4, BMPs, betacellulin, CNNs, cCAF, CTGF, CXC, CXCR3, Chemokins, cyr61, EGF, eNOS, epigen, epiregulin, FGF1-10, FSP-1, FGFR, Follistatin, GRO-α, GM-CSF, HGF, HGFL, HGH, HIF1-α, IGF-1, IL-1α, IL-1β, IL-10, iNOS, inhibitins, Interferons, KGF, Leptin, Lymphokins, MCP-1/CCL2, MIP2, MMP, MSP, Mullerian Inhibiting substance, NDF1-4, NGF, NO, neuregulin, Nodals, nov, PDGF, PLGF, PRGF, SF, Smad1-4, STAT1-3, TCRγ, TGF-α, TGF-β, TIMP, TNF-α, TNFRp55, t-PA, VEGF, WISP1-3, and combinations thereof.

61. The apparatus of claim 59 wherein the nucleic acid is in a dry form.

62. The apparatus of claim 61 wherein the nucleic acid is lyophilized.

63. The apparatus of claim 43 wherein the liquid is selected from the group consisting of saline, water, ethanol, a buffer, PBS, an acid, and an alkali.

64. The apparatus of claim 43 wherein the energy source generates the electric field based on pre-set parameters of duration, strength, and frequency of electrical pulses.

65. The apparatus of claim 43 wherein the energy source comprises a battery.

66. The apparatus of claim 43 wherein the energy source comprises an electric cord.

67. The apparatus of claim 43 further comprising a foot control device for actuating one or more of the energy source, the means for combining, and the means for delivering.

68. The cartridge of claim 1 wherein the bioactive agent comprises a macromolecule capable of treating tumors.

69. The cartridge of claim 1 wherein the bioactive agent comprises a macromolecule for inducing an immune response.

70. The cartridge of claim 1 wherein the bioactive agent comprises a macromolecule for enhancing an immune response.

71. The cartridge of claim 1 wherein the bioactive agent is selected from the group consisting of an antibiotic, a hormone, a drug, a steroid, and a vaccine.

72. The method of claim 26 wherein the tissue site is a tumor.

73. The apparatus of claim 43 wherein the bioactive agent comprises one of a chemotherapeutic agent, a dissolving agent, an herbal remedy, an antibiotic, an antifungal, an alopecia agent, a growth factor, a steroid, and an anti-viral agent.

74. The apparatus of claim 73 wherein the bioactive agent comprises a chemotherapeutic agent.

75. The apparatus of claim 43 wherein the bioactive agent comprises one of an immunogenic antigen, a vaccine, and an antibody.

76. The apparatus of claim 43 wherein the bioactive agent is selected from the group consisting of an antibiotic, a hormone, a drug, a steroid, and a vaccine.

77. The cartridge of claim 1 wherein the bioactive agent comprises a nucleic acid encoding KGF.

78. The method of claim 26 wherein the bioactive agent comprises a nucleic acid encoding KGF.

79. The apparatus of claim 43 wherein the bioactive agent comprises a nucleic acid encoding KGF.

80. A cartridge for the delivery of a composition to a tissue of a subject, comprising:
a first chamber comprising a bioactive agent comprising a nucleic acid encoding a growth factor or cytokine;
a second chamber comprising a liquid, the second chamber being separated from the first chamber by a modifiable membrane;
an injection needle extending from a distal end of the cartridge through which the composition is administered;
a conductive portion for transmitting an electrical pulse to the distal end of the cartridge;
a plurality of electrodes extending from the distal end of the cartridge and operatively connected to the conductive portion,
wherein an increase of pressure in one of the first chamber and the second chamber causes modification of the membrane, the modification of the membrane permitting mixing of the bioactive agent and the liquid to form the composition.

81. An apparatus for the delivery of a composition to a tissue of a subject comprising:
a cartridge comprising:
a first chamber comprising a bioactive agent comprising a nucleic acid encoding a growth factor or cytokine;
a second chamber comprising a liquid, the second chamber being separated from the first chamber by a modifiable membrane;
an injection needle extending from a distal end of the cartridge through which the composition is administered;
a conductive portion including electrical wires for transmitting an electrical pulse to the distal end of the cartridge;
a plurality of electrodes extending from the distal end of the cartridge arranged circumferentially around the injection needle and operatively connected to the conductive portion,
wherein an increase of pressure in one of the first chamber and the second chamber causes modification of the membrane, the modification of the membrane permitting mixing of the bioactive agent and the liquid to form the composition;
an energy source for generating an electric field based on pre-set parameters of duration, strength, and frequency of electrical pulses, wherein the electrical pulses are transmitted from the energy source to the tissue via the conductive portion and the plurality of electrodes;
a plunger for contacting by an operator at a proximal end of the cartridge, wherein contact of the plunger causes the increase in pressure in one of the first chamber and the second chamber and modification of the membrane, and wherein the contact causes delivery of the composition to the tissue through the injection needle.

82. A method for delivering a liquid composition to a tissue site in a subject comprising:
modifying a membrane between a bioactive agent and a liquid in contiguous chambers to cause solubilization of the bioactive agent by the liquid to form the liquid composition, the bioactive agent comprising a nucleic acid encoding a growth factor or cytokine, wherein the modifying is performed by increasing pressure in one of the contiguous chambers and rupturing the membrane;
administering the liquid composition to the tissue site in the subject immediately after the modifying step;
establishing an electrical field at the tissue site in the subject.

83. The apparatus of claim 43 further comprising a safety component for controlling the energy source.

84. The apparatus of claim 83 wherein the safety component is selected from the group consisting of a fuse, a circuit breaker, and a switch.

85. The apparatus of claim 83 wherein the safety component prevents the energy source from generating an electric field.

86. The apparatus of claim 83 wherein the safety component prevents the at least one electrode from delivering the electric field to the tissue.
